(19) United States
(12) Patent Application Publication
(10) Pub. No.: US 2019/0139638 A1
(43) Pub. Date: May 9, 2019

(54) METHOD AND APPARATUS FOR LABELING AND TRACKING MEDICINAL SUBSTANCES

(71) Applicant: CODONICS, INC., Middleburg Heights, OH (US)

(72) Inventors: Gary KEEFE, Brecksville, OH (US); Lawrence SRNKA, Northfield Center, OH (US); Ross GOODMAN, Solon, OH (US); Stephen MUCHER, Yellow Springs, OH (US); Edward KUBIEZ, North Ridgeville, OH (US)

(21) Appl. No.: 16/011,993

(22) Filed: Jun. 19, 2018

Related U.S. Application Data
(60) Provisional application No. 62/521,851, filed on Jun. 19, 2017.

(51) Int. Cl. G16H 20/13; G16H 40/20; G06Q 10/08

(52) U.S. Cl. CPC G16H 20/13; G06Q 10/087; G16H 40/20

(57) ABSTRACT

Provided are methods and systems for tracking a drug. Information associated with a drug is received from at least a code reader that scans a code associated with a drug container, and a drug server that receives information from a drug storage device storing an inventory of drugs. The drug container associated with the code scanned by the code reader is selected from the inventory of drugs. A result of a comparison of information included in: (i) the information obtained in response to reading the computer-readable code, and (ii) the portion of the drug removal information received from the drug server is obtained. Issuance of an alert is controlled based on the result of the comparison indicating that a conflict exists.

### BACKGROUND OF THE INVENTION

### 1. Field of the Invention

This application relates generally to a method and apparatus for labeling and tracking medicinal substances and, more specifically, to monitoring usage of medicinal substances at individual stocking locations to determine inventory levels and, more specifically, to aggregating information about the usage of medicinal substances at remote locations to manage inventory, maintain patient medical records, perform billing related functions and track the disposal or waste of drugs.

### 2. Description of Related Art

It is common for medicinal substances, such as drugs for example, to be stocked at multiple locations in a healthcare facility. Healthcare facilities, such as hospitals for example, frequently maintain many types of drugs at substantial inventory levels in operating rooms to support a variety of different surgical procedures.

Locations such as operating rooms are sometimes referred to as ORs. These are often at remote locations in the hospital relative to areas where drugs are stored such as the pharmacy for example. This can create a challenge for hospital personnel in monitoring, replenishing and maintaining the inventory of drugs in these remote locations.

A common technique to manage drug inventories in ORs is to establish the minimum amount of an item, such as a drug vial or ampoule for example, that must be in stock to meet demand for a given period, such as a day or a week. This minimum stock level for an item is sometimes called a PAR level. Items with inventory levels that are below the established PAR level are restocked so the total number of the item at that location equals or exceeds the PAR level.

A maximum stocking level can also be established that is greater than the PAR level.

Drugs stocked in the OR are commonly stored in bins or designated areas within a locked cart sometimes called an “anesthesia cart”. Some anesthesia carts are basic with pull out drawers, manual locks and limited automation. These are sometimes called “dumb carts”.

Another type of cart, sometimes called a “smart cart”, can have significant automation built-in to the cart with integrated computers, barcode scanners, RFID tag reading/writing equipment and other technologies that perform additional functions including electronically controlling the dispensing of some drugs. The electronically dispensed drugs typically include drugs designated as controlled substances. An example of a controlled substance is a drug or substance, the manufacture, possession, or use of which is regulated by a government (e.g., designated as Schedule II Controlled Substances, Schedule III Controlled Substances, as established in U.S. Drug Enforcement Administration regulations, 21 C.F.R. Sections 1308.11 through 1308.15).

Certain technologies, such as apparatus designed to read and/or write machine readable RFID tags for example, can be incorporated into the design of the cart to expand the inventory management capabilities of the cart beyond controlled substances. This allows other types of drugs and inventory items stored in the cart to be easily tracked by reading the individual RFID tags affixed to the items when they are inserted or removed from the cart. It should be noted that most drugs and other items stored in anesthesia carts are not designated as controlled substances and not managed and tracked by smart carts as strictly, but it is still necessary to account for the use of these drugs and items during medical procedures for patient records and other business purposes such as billing.

Maintaining inventory levels in dumb carts is usually done manually by counting the drugs in each bin or designated storage area within the cart drawers and restocking the drugs by adding the number of units necessary to meet or exceed the established PAR levels for those drugs. This process can be time consuming for healthcare personnel especially when repeated in a large number of remote locations such as hospitals with many ORs.

Maintaining inventory levels in smart carts usually relies on the automation features of the cart. For drugs that are designated controlled substances, the drug vials or ampoules are frequently dispensed by the cart individually per a clinician request using the automation features of the cart. This enables the cart to maintain an accurate count of each drug dispensed. Other drugs stored in the cart that are not controlled substances are typically not dispensed individually, but can be retrieved as needed. Clinicians commonly open the cart drawers and remove such drugs from their bin or storage areas in much that same way that drugs are removed from dumb carts. However, the clinician is expected to use the automation features of the cart to record the removal of the drug by manually entering the drug information into the cart computer or scanning the barcode on the label of the drug vial or ampoule that identifies the drug using a barcode scanner that is part of the automation system of the cart to update the inventory of the drug.

In the case of smart carts that make use of RFID technology, the cart can detect drugs and other inventory items affixed with RFID tags are inserted in, or removed from, the cart drawers in near real-time by reading the individual RFID tags of the items contained in the drawers of the cart whenever the drawer is opened and then closed. The RFID tags of items added or removed from the drawers can be quickly identified allowing an accurate accounting of RFID tagged items in the cart to be determined.

Because operating rooms are often high stress environments where clinicians are frequently challenged to deliver patient care, omissions in recording drugs removed from the anesthesia cart, such as a failure to scan drugs with the barcode scanner connected to smart carts, can occur and result in inaccurate inventory reporting on the cart automation system. When technologies that enable automatic inventory identification and accounting, such as RFID for example, are not employed in the cart, manual inventory checks are commonly used to ensure accurate stock levels are maintained. This can negate some of the benefits of smart carts.

Once drugs are removed from either a dumb cart or a smart cart, the drugs are typically prepared one-at-a-time by transferring the drug from each vial or ampoule into another container, such as a syringe for example, that is labeled with information about the drug in its final form in the syringe.

The process of creating the label for the syringe commonly requires scanning the NDC code encoded in the barcode on the manufacturer's label of the vial or ampoule on a drug labeling device that is separate from, and operates independently of the anesthesia cart to create the label. The NDC is the National Drug Code used in the U.S. to identify the drug in a container. The drug labeling device identifies the drug from the NDC code and prints a secondary label with the required information that is to be affixed to the syringe to properly identify the drug contained in the syringe.

Although the anesthesia cart and the drug labeling device operate independently of each other, the drug labeling device can be physically attached to (e.g., rests on top of), or is positioned in close proximity to, the anesthesia cart. The proximity of the drug labeling device to the anesthesia cart and the clinical function it performs of printing the syringe labels required by federal regulation promotes high compliance by clinicians in scanning drugs removed from anesthesia carts.

The high level of compliance by clinicians in scanning the barcode of the drug vial or ampoule on the drug labeling device to generate the secondary label is driven by regulatory requirements for printing a label with proper drug identification when the drug is transferred to a dispensing container such as a syringe, for example. This is in contrast to the lower compliance of scanning the vial on the automation system of the smart cart for inventory management, especially if the use of more automatic inventory identification technologies such as RFID for example are not employed, because that activity can sometimes be a distraction from the task of delivering patient care in the OR, and may be deemed unnecessary by some clinicians because of the less stringent monitoring of drugs that are not controlled substances.

### BRIEF SUMMARY OF THE INVENTION

Accordingly, there is a need in the art for a method and apparatus for recording drug inventory usage to maintain proper inventory levels at remote locations in healthcare facilities without interfering the with normal workflow of clinicians engaged in the delivery of health care.

According to one aspect, the subject application involves a remote barcode reader that is to communicate with a terminal in a medical network. The remote barcode reader includes an optical barcode scanner that transmits a signal indicative of a barcode in response to interrogating the barcode. A non-transitory computer-readable memory stores, at least temporarily, information obtained in response to reading the barcode. A network interface communicates wirelessly over a wireless communication channel with a remote device in a medical network to obtain information pertaining to a drug that is identifiable from the information obtained in response to reading the barcode. A processor initiates the transmission of a communication based on the information obtained from the barcode to the remote device, and delays transmitting at least a portion of the information obtained from the barcode until at least a time when a response including information related to the drug is received from the remote device.

According to another aspect, methods and systems for tracking a drug are provided. Information associated with a drug is received from at least a code reader that scans a code associated with a drug container, and a drug server that receives information from a drug storage device storing an inventory of drugs. The drug container associated with the code scanned by the code reader is selected from the inventory of drugs. A result of a comparison of information included in: (i) the information obtained in response to reading the computer-readable code, and (ii) the portion of the drug removal information received from the drug server is obtained. Issuance of an alert is controlled based on the result of the comparison indicating that a conflict exists.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

The invention may take physical form in certain parts and arrangement of parts, embodiments of which will be described in detail in this specification and illustrated in the accompanying drawings which form a part hereof and wherein:

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows an illustrative embodiment of a computer terminal for drug labeling;

FIG. 2 schematically shows an illustrative embodiment of components included as part of the embodiment of the computer terminal shown in FIG. 1;

FIG. 3 shows an illustrative arrangement of a communication network at a healthcare facility;

FIG. 4 shows an illustrative embodiment of a drug cart and drug cart controller provided with a barcode scanner;

FIG. 5 shows an illustrative embodiment of a drug labeling system in communication with a drug cart and drug cart controller provided with a RFID tag reader;

FIG. 6 shows an illustrative embodiment of a method of tracking a drug removed from a drug cart; and

FIG. 7 shows another illustrative embodiment of a method of documenting drug waste.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

Attempts to improve automated monitoring of drug inventory levels in a drug cart based on information from a drug labeling system has failed, in part, because drug carts and labeling systems operate independently of each other. RFID drug labeling techniques traditionally been incompatible with traditional drug labeling techniques that utilize a machine-readable code other than a RFID tag. As a result of such an incompatibility between coding standards, there has been an inability to use a combination of drug cart data and labeling data to track drug consumption beyond indicating the presence of a number of containers of each drug within a drug cart.

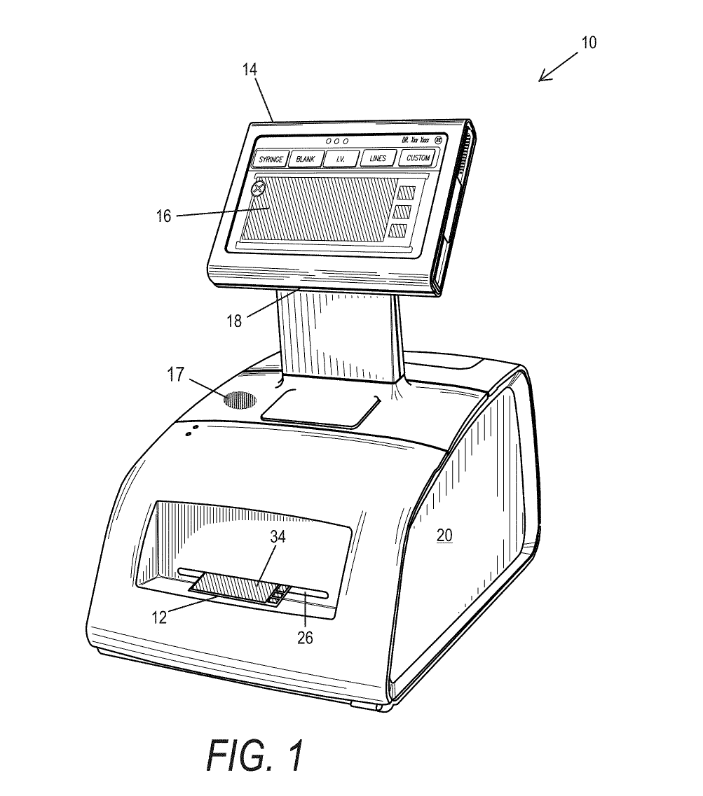

As shown in FIG. 1, the computer terminal 10 includes a touch-screen display 14 that can be pivotally coupled to a cabinet 20 to display a virtual label 16 comprising label content 34 that will be printed onto a label 12 that will be applied to a medicinal substance. The computer terminal 10 can be operable to scan a computer-readable code and print a label to be applied to a medical container such as a syringe as described in U.S. Pat. No. 9,262,585 to Keefe et al., which is incorporated by reference herein in its entirety. The display 14 can display soft keys that, when touched by a technician or any other user, inputs data and commands into the computer terminal 10. The virtual label 16 is a computer-generated rendering of the label 12 that offers the user visual confirmation of the appearance of the physical label 12 to be printed by a printer 26. A computer-input peripheral such as a non-contact scanning system 18 can be provided at a convenient location, such as integrally formed in a bottom portion of the display 14 to read a machine-readable code supported beneath the scanning system 18 for example. Integrally forming the components of the scanning system 18 as part of the display 14 or the cabinet 20 provides for space savings over an arrangement where the components of the scanning system 18 are formed as one or more separate peripherals, which can be repositioned relative to the display 14 and/or cabinet 20. However, other embodiments can allow for a separate and distinct components of the scanning system 18, display 14 and/or cabinet 20.

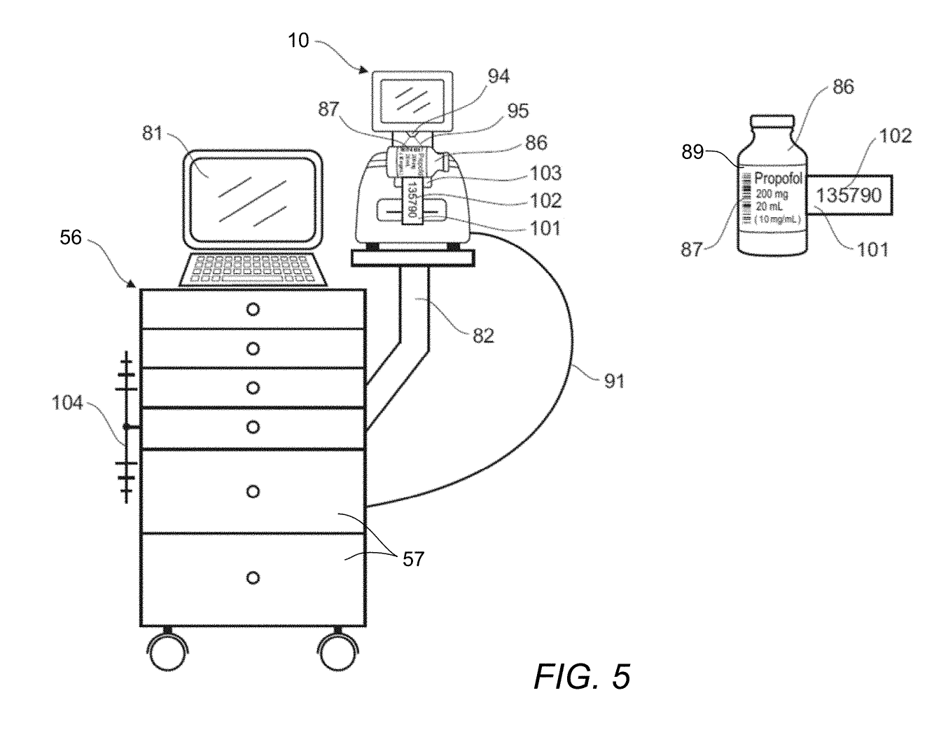

The computer-input peripheral can be a barcode reader or radio-frequency identification (“RFID”) tag reader, or any other device that reads a machine-readable code such as a barcode or RFID code, respectively, or any other machine-readable code without requiring contact between the computer terminal and the code, and optionally the user during entry of the code. Embodiments of the scanning system 18 can include both a barcode reader that uses an optical scanning technology to read a pattern of printed markings and a RFID tag reader. For embodiments such as that illustrated in FIG. 5, the barcode reader 94 can be provided adjacent to, and optionally integrated within the underside of the display 14. The RFID tag reader 103 can optionally be housed within the cabinet 20, and positioned to interrogate a RFID tag 101 provided to a drug vial 86 as a barcode 87 on a label 89 applied to the drug vial is read by the barcode reader 94, as shown in FIG. 5. However, one or both of the barcode reader 94 and the RFID tag reader 103 can be external peripherals, plugged into compatible ports (e.g., USB port, etc.) of the computer terminal 10, optionally to be used separately. In other words, the barcode reader 94 and the RFID tag reader 103 are not necessarily positioned so that a barcode and a RFID tag can be read with the vial 86 positioned at one location. According to alternate embodiments, the display 14 can be utilized by a user as the computer-input peripheral. For such embodiments, the soft keys displayed by the display 14 can be selected to input information such as a medicinal substance being prepared to be administered to a patient or other information to be utilized in generating the label as described herein. According to yet alternate embodiments, a speaker 17 can optionally be provided to the display 14 or any other portion of the computer terminal 10 to broadcast audible sounds.

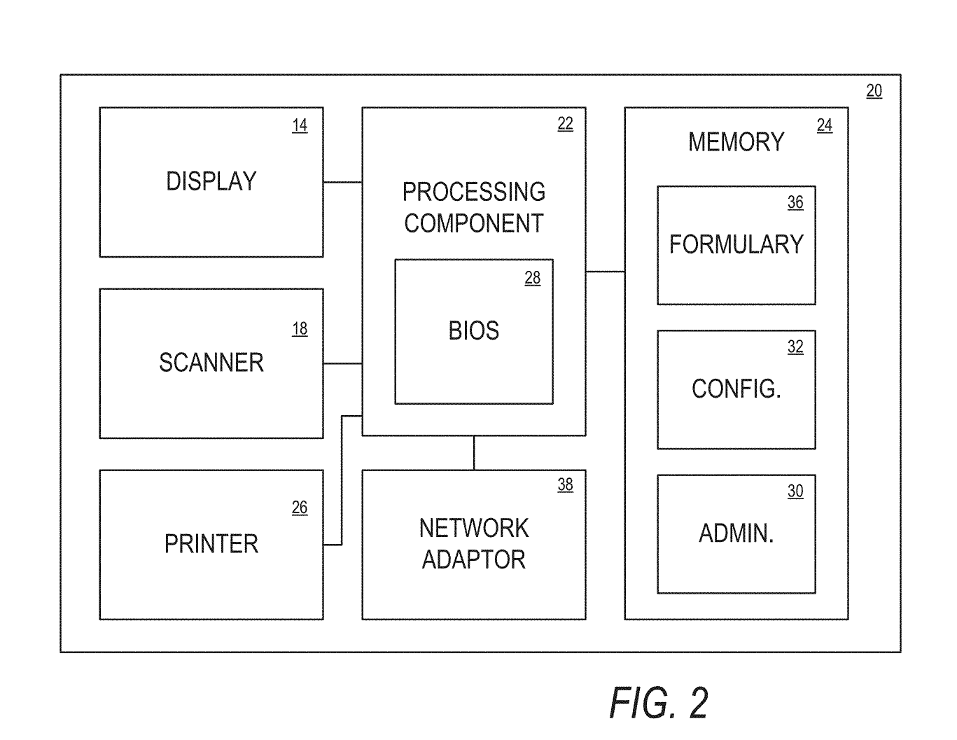

The computer terminal 10 also includes a cabinet 20 that houses or supports components that are operable to produce the label 12 in compliance with a medical labeling standard. But if what is being labeled is anything other than the medicinal substance, then the label 12 produced is to be compliant with a standard developed by a trade or professional organization, governing body, government agency, a healthcare provider or facility such as a hospital, or any other standards body setting forth policies for labeling such material. The internal components housed within the cabinet 20 are schematically illustrated by the block diagram of FIG. 2. The components can be formed from an arrangement of computer hardware such as ASICs, computer processors, programmable logic controllers and other circuitry; or a combination of computer hardware and computer-executable instructions. For example, a processing component 22 is provided to execute computer-executable instructions stored in a non-transitory, computer-readable memory 24 such as a hard disk drive, read-only memory (“ROM”), random access memory (“RAM”), optical disc, or any other suitable memory device, or any combination thereof. The computer-executed instructions, when executed by the computer processor 22, result in the performance of the method of generating a label for a medicinal substance described in detail below. A BIOS 28 is provided to load the operating system and other such administrative instructions 30 stored in the memory 24 and manage hardware interface permissions of the computer terminal 10. The operating system can be configured to only load authorized updates to prevent unauthorized changes to a formulary 36, configuration data 32 and administration instructions 30. Configuration data 32 controls various features of the computer terminal 10 that are active and available for use at any given time. The configuration data 32 can optionally be stored, updated and deleted from the memory 24 by the introduction of a so-called smart drive comprising a USB compatible flash memory to the computer terminal 10. When the smart drive is introduced to the computer terminal 10, it establishes the configuration data 32 of the computer terminal 10. The configuration data 32 can optionally be used to deactivate functional features that the computer terminal 10 would otherwise be able to perform based on the model of the computer terminal 10 purchased. Accordingly, a common hardware platform of the computer terminal 10 can be configured in a plurality of different functional configurations based on the configuration data 32.

In addition to the administrative instructions 30, the memory 24 also stores an updatable formulary 36 containing a database of medicinal substances that can be identified by the computer terminal 10 and select information for each medicinal-substance entry in the database. The formulary 36 can optionally be stored, updated and deleted from the memory 24 by the introduction of a so-called smart drive comprising a USB compatible flash memory to the computer terminal 10. When the smart drive is introduced to the computer terminal 10, it establishes the formulary 36 of the computer terminal 10. Illustrative examples of the select information that can be provided for the medicinal-substance entries includes, but is not limited to, an ID number such as a National Drug Code (“NDC”), UPC code, EAN code, or any other identifying data that can be used to relate a barcode or other computer-readable code to the medicinal-substance entries; a sound file that, when played, audibly announces the name of the medicinal substance identified in response to scanning a machine readable code; warning data; designations of drugs corresponding to the entries as controlled substances (e.g., narcotics, substances with potentially serious effects, etc.), or any combination thereof. As noted above, controlled substances can include narcotics and other Schedule II and Schedule III substances under the U.S. Drug Enforcement Administration regulations, 21 C.F.R. Sections 1308.11 through 1308.15. However, as used herein, controlled substances can also include drug or other substance that, although not classified as a Schedule II or Schedule III substance, has the potential for abuse as a recreational drug, requires limited and/or controlled administration to patients, could potentially harm a patient if administered in excessive quantities, etc. Thus, the respective formulary entries for any drugs and other substances can be customized to include data designating those drugs and other substances as controlled substances.

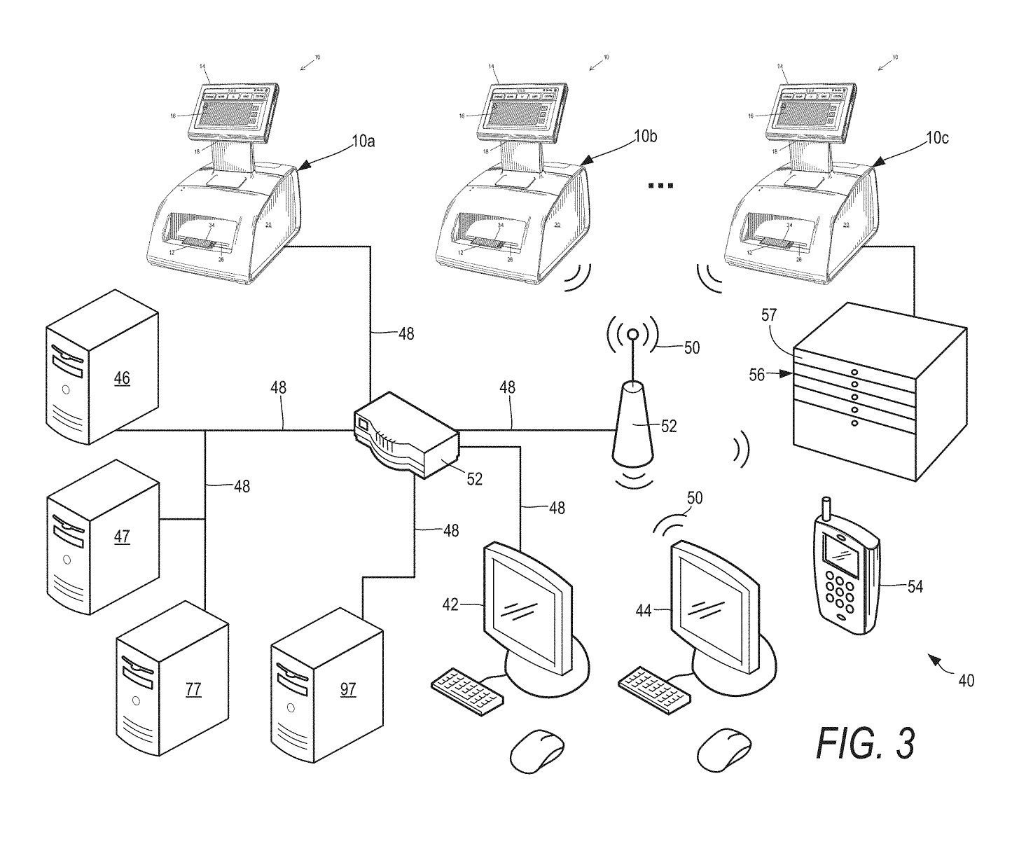

Embodiments of the formulary 36 can also optionally include quantity data associated with one, a plurality or each of the drugs in the formulary 36. The drugs having a field indicative of the number of single use vials, for example, remaining in a certain drug cart 56 associated with the computer terminal 10c, as shown in FIG. 3, can optionally be monitored by the computer terminal 10 and/or a remote terminal such as a pharmacy terminal 42 described below, for example, to ensure a sufficient supply of those drugs is available from the cart 56. According to one embodiment, drugs to be monitored can be associated with a minimum threshold field that indicates the minimum quantity of single use vials, for example, that must be stored by the cart 56 as a minimum inventory, as established on a case-by-case basis by the health-care facility where the cart 56 is located. Similarly, another field is associated with a number indicating the actual number of single use vials of at least a portion, and optionally each drug present in the cart.

A network adaptor 38 is operatively connected to communicate with the processing component 22 for translating signals received by the computer terminal 10 over a network 40 at a medical facility, such as that illustrated in FIG. 3. The network adaptor 38 can be compatible with any type of network communication. For example, the network adaptor 38 can include a hardwired, 10Base-T, 100Base-T, or 1000Base-T Ethernet interface with an RJ-45 socket, a coaxial cable interface, a fiber-optic interface, any format of wireless communication interface such as an antenna compatible with any of the 802.11 standards established by the IEEE, or any combination thereof. Embodiments including wireless network adaptors 38 can employ any desired securing protocol such as WEP, WPA and WPA2, for example, and other suitable security protocol. For embodiments including a network adaptor 38 compatible to communicate over a plurality of different network communication channels, both a hard-wired communication portion of the network adaptor 38 and a wireless communication portion of the network adaptor 38 can optionally be concurrently active. Thus, the computer terminal 10 can optionally communicate via both the hard-wired and wireless portions of the network adaptor 38 concurrently.

As shown in FIG. 3, a plurality of the computer terminals, each referred to generally at 10 and separately at 10a, 10b, 10c, can be included in a network 40 at a healthcare facility. For example, each operating room in which surgical procedures take place may have one of the computer terminals 10 located therein. Other networks may include a computer terminal 10 in an examination room where procedures such as minimally invasive examinations of patients are conducted. Each of the computer terminals 10a, 10b, 10c can be provided with a unique identifier, which can be stored electronically in the memory 24 (e.g., as part of the configuration data 32 or administration data 30, or separately from such data, etc.), to uniquely identify those terminals 10a, 10b, 10c. The identifier of each terminal 10a, 10b, 10c can also optionally be stored in association with the respective location of those terminals, allowing each of the terminals 10a, 10b, 10c to be allocated to a particular OR.

The network 40 also includes a pharmacy computer terminal 42 executing computer-executable instructions (referred to hereinafter as an administration tool or “AT”) that, when executed, manage one or more, and optionally all of the computer terminals 10. Each computer terminal 10 to be managed by the AT can be optionally assigned a user-specified designation using the AT to distinguish the computer terminals from each other on the network 40, and to optionally provide the user with a brief description of each computer terminal 10. For example, a computer terminal 10 located in operating room #1 can be assigned the designation OR-1 to indicate its location. According to alternate embodiments, the user-specified name Cart-1 could be assigned to a computer terminal on mobile cart #1. An IT computer terminal 44 can also optionally be connected as part of the network 40 to execute the AT and allow technical personnel to manage technical aspects of the computer terminals 10, but optionally exclude from the permissions granted to technical personnel the ability to alter drug or other medical-related content stored by the computer terminals 10. The permissions granted to a user at the terminals 42, 44 can optionally be determined when the user logs in based on a username/password combination, a computer-readable identification, or any other identifying information. Thus, the terminals 42, 44 do not necessarily have to be dedicated solely for any particular purpose.

The pharmacy terminal 42 can be located in a pharmacy at a healthcare facility, where an inventory of controlled drugs and medicinal substances (hereinafter generally referred to as “drugs”) is maintained. A pharmacist or a plurality of pharmacists maintain and administer a master drug database (“MDD”) containing an identity, identification code (e.g., NDC) number, concentration, designation as a controlled substance, and other pertinent information for drugs used by the pharmacy. Drugs are entered into the MDD by the pharmacist, and the terminals 42, 44, and optionally other terminals connected to the network 40 can restrict access to the MDD and prevent unauthorized individuals from entering or altering drug entries in the MDD, and optionally from accessing the MDD altogether. In other words, the pharmacist(s) registered and authorized to work at the health care facility and those they grant permission to access the MDD are the only individuals permitted to manipulate data in the MDD.

From the MDD, the pharmacist manages a formulary to be stored in the memory 24 of one or more of the computer terminals 10 using the AT with the pharmacist permission. The formulary can include a subset, but less than all of the entries in the MDD, and the subset can optionally comprise drugs that are commonly used in the operating room or other locations at the healthcare facility where the computer terminal 10 is positioned. The formulary entries can include much of the same information included in the corresponding entry in the MDD including, but not limited to, the identification number (e.g., NDC, hospital ID, RFID unique ID, etc.), concentration information, dose information, volume information, the designation as a controlled substance, etc. The same formulary can optionally be stored in the memory 24 of more than one computer terminal, and can optionally be customized to include drugs utilized during surgical procedures relating to a particular medical discipline. For example, the same formulary comprising drugs commonly used during cardiac surgical procedures may be stored in the memory 24 of computer terminals 10a, 10b, which are each located in a respective operating room dedicated for such procedures. Another, different formulary comprising drugs, optionally in appropriate doses, suitable to be administered to children can be stored in the memory 24 of a computer terminal 10 located in an operating room dedicated for pediatric surgical procedures. According to alternate embodiments, the formulary 36 stored in the memory 24 of a computer terminal 10 can be evaluated and updated, replaced or otherwise changed before each surgical procedure if the operating room where the computer terminal 10 is located is not dedicated for a particular type of surgical procedure. By way of example, a subset, but fewer than all of the drugs in the MDD designated as suitable for use with children can be made selectable using the computer terminal 10 during a pediatric surgical procedure. When a formulary update is needed to accommodate a specific type of procedure, a pharmacist's access can be required to update, replace or otherwise change the formulary in the computer terminals 10, and updating, replacing and changing the formulary in the memory 24 in each of the computer terminals 10 can be performed over the network as described in detail below.

In addition to a pharmacist's level of permission, there can be other permission levels limiting access to the computer terminals 10 to different users. For example, an anesthesiologist may be granted permission to use a computer terminal 10 to interrogate a barcode or other machine-readable code on a drug vial to extract the identity of the drug and print a label to be applied onto a syringe. The anesthesiologist can optionally also be granted permission to enter confirmation into the computer terminal 10, indicating that the interrogation of a barcode has returned the proper drug identification. The formulary and/or MDD entry corresponding to the now-confirmed drug can be updated by the anesthesiologist to indicate that the drug identified by the corresponding machine-readable code is accurate, and such confirmation can optionally be shared over the computer network 40 to at least one additional computer terminal. However, the anesthesiologist may be prevented from editing the formulary stored in the memory 24 of the computer terminal 10.

Additionally, an IT professional can be granted permission to address any technical, computer hardware-and-software-related issues with the computer terminals 10 that are unrelated to the specific drug information of the MDD and/or formulary. For example, the IT professional may be granted permission to assign and/or change: an IP address of the computer terminals 10, a security protocol employed, and other computer-specific matters. However, some information related to the formulary such as the version and description of the formulary can be viewed by the IT professional to ensure that the proper computer terminal 10 has the correct formulary installation. This also applies to version and description information of the operating system, BIOS 28, configuration data 32 and administration instructions 30.

The network 40 in FIG. 3 also includes a drug cart server 46 that communicates a drug cart 56 inventory system at the healthcare facility. The drug cart server 46, like the computer terminals 10, and optionally other resources of the network 40, can receive signals to other network communications via hard-wired communication channels (represented by solid lines 48 in FIG. 3) such as CAT-5 or CAT-6 Ethernet cable, via wireless communication channels (represented by arched, radiating signals 50), or a combination thereof. For example, the drug cart 56 can optionally include a RFID tag reader 104, as shown in FIG. 5, positioned to emit an alternating radio-frequency (“RF”) electro-magnetic field that reaches the interior of each drawer 57 monitored using the RFID tag reader 104. Each vial 86 containing a drug can be provided with its own RFID tag 101 encoding a unique RFID tag code 102. Being unique, the RFID tag code 102 uniquely identifies each individual vial 86 from the others in the drug cart 56. By uniquely identifying each vial 86, the RFID tag code 102 can distinguish between vials 86 containing the same drug, but in different concentrations, or that formed parts of different lots, for example, instead of generically identifying just the name of the drug contained by each vial 86. The RFID tag 101 can optionally be a passive tag, which includes an antenna in which an electric current is induced by an alternating RF electro-magnetic field emitted by the RFID tag reader 104. The induced electric current energizes a circuit operatively connected to a memory on board the RFID tag 101, causing the circuit to transmit the unique RFID tag code 102 stored in the memory on the RFID tag 101 in response. As an example, the RFID tag code 102 can be transmitted by the RFID tag 101 by modulating the alternating RF signal emitted by the RFID tag reader 104. The code transmitted by the circuit of the RFID tag 101 is received by the RFID tag reader 104 and conveyed to a drug cart controller 81, which can optionally interpret and store the received RFID tag code 102 and transmit the received RFID tag code 102 via the network 40 to the drug cart server 46.

The drug cart server 46 can optionally be server hosted for, or on behalf of the healthcare facility, or can be a centralized server hosted by a third party that supports a plurality of drug cart servers 46 at different healthcare facilities. The drug cart server 46 can be configured with computer-executable instructions that, when executed, cause the drug cart server 46 to identify the vial 86 based on the RFID tag code 102. For example, the drug cart server 46 can optionally be provided with a server formulary that is referenced to identify the drug in the vial 86, along with the pertinent information concerning the identified drug, such as concentration information, expiration information, etc.

Network communication equipment 52 such as switches, routers, wireless access points, private and/or public communication channels (e.g., switched public-telephone lines and CAT-5 or CAT-6 Ethernet cable utilized by the Internet, a local area network, etc.), and the like can be utilized by the network 40 to share network resources and establish communication between the computer terminals 10 and the terminals 42, 44. Additionally, the computer terminals 10 can optionally serve as an expansion port to which other network resources such as the automated drug dispensing system 56, interchangeably referred to herein as a drug cart 56, commonly referred to as a “smart cart”, can be connected to the network to dispense and document the strength, quantity and type of drug according to a schedule or in response to the occurrence of a predetermined event. Additionally, since one of the functions of smart carts is to control the dispensing of drugs and one of the functions of computer terminal 10 is producing labels for containers such as syringes that are filled with drugs from the smart cart, there are benefits related to efficiency if the devices can share information. For example, a network connection between the smart cart and computer terminal 10 will allow user login information such as username and password entered on one device to be shared with the other device so a user is authenticated on both devices with a single login. Other benefits include being able to share information about drugs being used in a procedure between the devices so verification and reconciliation of drugs can be performed to ensure the proper medications are dispensed, labeled and tracked for improving the accuracy of patient records and accurate billing. As shown in FIG. 3, the automated drug dispensing system 56 can be hard-wired via a cable connection 91 to the computer terminal 10c, which is connected wirelessly to other network resources, but the automated drug dispensing system 56 can also optionally be configured to communication with the computer terminal 10c indirectly, through devices collectively making up the network 40.

As a specific example of the information shared between the computer terminal 10c and the smart cart 56 is drug consumption information. According to such an example, when information identifying a controlled substance (e.g., NDC) is entered into the smart cart 56 when such a controlled substance is to be removed and administered to a patient, information identifying that controlled substance can be transmitted to the computer terminal 10c. The transmitted information can be used by the computer terminal 10c to prepare and generate a label to be applied to a syringe acting as a delivery container for the controlled substance. However, the computer terminal 10c can also update a log stored in the memory 24 of drugs consumed in that specific OR in which the computer terminal 10c is located (and/or from that specific cart 56). For instance, when the controlled substance is accessed and obtained from the smart cart 56, the information identifying the drug entered to the smart cart 56 to unlock a secure drawer 57 (FIG. 3) of the cart 56 storing the controlled substance, or otherwise grant a clinician access to the drug is transmitted to the computer terminal 10c via the network 40 or via a local, hardwired connection. This information can be transmitted to the computer terminal 10c according to a predetermined workflow of the cart 56 and/or controller 81, or can optionally be transmitted to the computer terminal 10c in an effort to identify the drug that was not identifiable by the cart 56 (e.g., by the controller 81). At least a portion (but less than all), and optionally all of the information transmitted to the computer terminal 10c can be used by the computer terminal 10c to identify the drug and/or generate the label as described herein for labeling a syringe. Also based on the transmitted information, the computer terminal 10c can update a log in the memory 24 with consumption information that can be used to determine that a quantity of the controlled substance was removed from the smart cart 56. This consumed quantity can then be transmitted by the computer terminal 10c via the network 40 to the pharmacy terminal 42 or other suitable destination where inventory information for that OR is maintained (e.g., the controller 81 provided to the cart 56 as described below). Should the inventory of the controlled substance fall below a threshold value (e.g., the PAR value), the pharmacy terminal 42 can issue an alert to an appropriate party indicating that the controlled substance in the smart cart 56 should be replenished above the threshold value.

As noted above, however, medicinal substances that are not controlled substances (referred to hereinafter as “uncontrolled substances”) are often accessible from the smart cart 56 without entering access information identifying those uncontrolled substances as a condition for granting the clinician access to such drugs. The entry of such information is not typically mandated by or used to assist in the compliance with any regulations applying to controlled substances. However, a delivery container such as a syringe containing such uncontrolled substances is required by the law of a sovereign government or other governing-body regulations to be labeled. Thus, when such uncontrolled substances are removed from the smart cart 56, there may be no information to transmit to the computer terminal 10c if the clinician has elected not to voluntarily enter such information. However, when the computer terminal 10c is used to prepare and generate the label for an uncontrolled substance as described herein, the computer terminal 10c can update the log of consumption information stored in the memory 24 (or a computer-readable memory provided to the cart 56, for example) to reflect the consumption of the uncontrolled substance despite not receiving transmitted identifying information from the smart cart 56. Instead, the information obtained by the computer terminal 10c in response to using the scanner 18 to read a computer-readable code (e.g., NDC on the vial label) as described herein can be utilized to keep a running log of drug consumption based on the assumption that the drug being labeled was obtained from the cart 56. The computer terminal 10c can utilize the same or similar workflow when preparing labels for any uncontrolled substances that were not identified to the cart 56, as well as substances (controlled substances or non-controlled substances) obtained from a so-called “dumb cart” that simply allows the manual retrieval of all drugs, including controlled substances, without entry of the drug identifying information to that cart. Thus, for smart carts 56 that lack an inventory capability for all drugs and dumb carts, the computer terminal 10c can allow for the maintenance, in real time as the drugs are consumed, or at designated periods of time that are allotted for the individuals tasked with maintaining the inventory of drugs in the carts, information pertaining to the remaining stock of drugs in such carts.

Although the embodiments above involve entering the NDC to identify the drug to the cart 56 and scanning a computer-readable code encoding the NDC with the scanner 18 to identify the drug, the present disclosure is not so limited. Alternate embodiments can entail entering any suitable information to uniquely identify a controlled substance to be accessed and removed from the cart 56 during a medical procedure, and reading any computer-readable code encoding any suitable information that allows a drug to be uniquely identified. For example, a proprietary code specific to the hospital or other health-care institution, private labeling standard, or other entity that is not subject to the control of a governmental or professional regulatory body can be used without departing from the scope of the present disclosure. An example of such alternate information or codes includes a hospital identification number (“HID”) used for internal purposes within a hospital or other healthcare facility. The HID can optionally be utilized elsewhere within the hospital or other facility to refer to the drugs in question, such as within an electronic medical record (“EMR”) system, billing system, and/or other system within the hospital or facility for purposes of managing the financial, business, and/or administrative aspects of providing healthcare.

The formulary 36 can also optionally include a field, value or other attribute for each drug or other substance having an entry in the formulary that indicates whether a label is to be printed for those respective entries. According to alternate embodiments, instead of a dedicated field indicating whether a label is to be printed, the memory 24 can store “null” values for the label information as a signal that a label is not to be printed for that entry, or any other suitable indication that, when referenced by the computer terminal 10c, instructs the computer terminal 10c to forego printing a label for the scanned substance. For example, eye drops may be administered to patients as part of certain medical procedures. However, eye drops that purely moisturize the eyes to minimize irritation of the eyes while the patient is sedated do not require a label to be printed and applied. Instead, the bottle of eye drops, which may already bear a label, is simply removed from the cart 56 and the eye drops administered to the patient. However, to aid in the monitoring of the cart's inventory, clinicians may be encouraged to scan a computer-readable code (e.g., barcode) on the bottle of eye drops before, after or during the medical procedure during which the eye drops are used. In response to reading this computer-readable code with the scanner 18 provided to the computer terminal 10c, the computer terminal 10c references the formulary 36, specifically the field indicating whether a label should be printed for this entry, to determine that a printed label is unnecessary for this substance. The computer terminal 10c creates, stores or updates the information evidencing consumption of the scanned eye drops logged in the memory 24 by the computer terminal 10c, but does not print or otherwise produce a hardcopy of the label as it does for labeling syringes containing other substances elsewhere herein.

In addition to the drug formulary 36, the memory 24 or other computer-readable medium accessible to the computer terminal 10c, locally and/or remotely over the network 40, can also optionally store another database with entries for non-drug items (referred to as “tools”) consumed or used in the OR where the computer terminal 10c is located. For example, syringes, gauze, intravenous lines, etc. may be stocked in the cart 56 and used during a medical procedure in the OR. As such, their supplies in the cart 56 must be replenished when they fall below a threshold level to ensure their availability during subsequent medical procedures in that OR. Unlike the formulary 36 of drugs storing NDC-compliant data for drugs subject to NDC rules and regulations, the tools having entries in the additional database can be any miscellaneous object other than a drug having an identifier assigned by a regulatory body, such as a NDC for example. Since the tools lack a NDC, the computer-readable code can be a UPC code, EAN code, or any other computer-readable code that uniquely identifies the tools. The computer terminal 10c can be configured with computer-executable instructions stored in the memory 24 to refer to this additional database when a computer-readable code is scanned to document the usage and/or consumption of the tools for purposes of monitoring the inventory of the cart 56. When the inventory of the tools available from the cart 56 falls below acceptable levels as defined by the facility or other party affiliated with the facility where the cart 56 is located, the log of inventory information transmitted by the computer terminal 10c can be used to determine what stocks need to be replenished, when, and the location of the cart 56.

Also, since the computer-readable code provided to, associated with, or otherwise used to identify the tools is not a NDC or other code compliant with a drug-labeling standard, the accuracy of such codes may not need to be verified as described herein to grant access to the tools in the cart 56. However, once the identity of the tool identified based on the scanning of the computer-readable code has been verified as accurate, such verification can be made available to one, a plurality, or each of the computer terminals 10 connected to the network 40. As an alternate embodiment, the verification of the accuracy of the tool identity based on the computer-readable code can be skipped at a time when the tool is accessed to be used during a medical procedure in the OR. The skipping of such verification can be recorded by the computer terminal 10c affiliated with the cart 56 so the identity of the tool can be revisited and later verified after a time when the tool is accessed for use. Again, verification can be shared over the network 40 for use by any of the connected computer terminals 10.

Embodiments of the computer terminal 10c can optionally combine and/or reconcile consumption data transmitted by the cart 56 and consumption data obtained by the computer terminal 10c in response to scanning a computer-readable code using the scanner 18 to arrive at a more-accurate inventory level in the cart 56. Thus, consumption of both controlled and uncontrolled substances can be accounted for. The inventory information indicative of the remaining drug stock can optionally be transmitted via the network 40 to a suitable destination where decisions regarding the replenishment of the drugs in the cart 56 can be made (for example, the pharmacy terminal 42, the controller 81 described below, etc.). Information pertaining to the updated log can optionally be transmitted by the computer terminal 10c in real time as the labels are generated, in batches such as following the conclusion of a surgical procedure or at the end of each day, or in any other desired manner to a desired destination to signal a need for drug replenishment.

The embodiment described above describes the computer terminal 10c maintaining an inventory of drugs stored by the cart 56. However, according to alternate embodiments, the inventory of drugs remaining in a cart can optionally be maintained by a computer terminal (e.g., the pharmacy terminal 42) remotely located from the computer terminal 10c, but accessible for communications from the computer terminal 10c over the network 40. For such embodiments, the consumption information can optionally be transmitted by the computer terminal 10c in real time as the labels are generated, in batches such as following the conclusion of a surgical procedure or at the end of each day, or in any other desired manner. The pharmacy terminal 42 or other recipient of the consumption information can be programmed to update the log of drugs consumed at a central location. The same, or additional log can optionally be updated for each of a plurality of computer terminals 10a, 10b, 10c located in different ORs, for example, and issue an alert when the remaining stock of a drug falls below a threshold value or provide information about the consumption of drugs upon request over the network 40 to a remote computer terminal such as the pharmacy terminal 42. In response to the issuance of such an alert, the pharmacist or other suitable party can replenish the drug(s) that are in short supply.

The alert issued to the pharmacist or other party who is responsible for replenishing the low-quantity drugs in a cart 56 can optionally include a replenishment confirmation option. Once an order for at least partial replenishment of the low-quantity drugs has been issued, the responsible party can select the appropriate replenishment option via a user interface presented by the pharmacy terminal 42, for example, indicating that a certain quantity of the low-quantity drug is to be replenished in the cart 56. The certain quantity can optionally be a predetermined number of single use vials to bring the number of vials in the inventory in excess of the minimum threshold quantity (e.g. PAR level), the quantity required to fully replenish the low-quantity drug(s), etc. Such a confirmation can optionally be transmitted for each low-quantity drug being replenished. When the replenishment confirmation is issued, the pharmacy computer 42 or other terminal from which such confirmation is sent can transmit information over the network 40 to the affected computer terminal(s) 10 or otherwise update the appropriate fields in the formulary 36 or other database (e.g., centrally maintained database for a computer terminal 10). This information can notify the affected computer terminal(s) 10 that the stock has been replenished so future drug consumption from the cart 56 can be accurately maintained by the corresponding computer terminal 10c.

According to alternate embodiments, the clinician replenishing the stock in the cart 56 can optionally manually enter the quantity of drugs being deposited in the cart 56 into the computer terminal 10c. Regardless of how the restocking information is conveyed to the computer terminal 10c or other database, a reporting component can be utilized to generate reports documenting the drug consumption and/or restocking information. For example, the reports can outline the quantity of drug(s) consumed and/or restocked, the locations where the drugs stored and require restocking, the times at which the drugs were consumed and/or restocked, the patients to whom the drugs were administered during a medical procedure, etc. for audit purposes.

For many of the embodiments above, the information pertaining to the consumption of drugs and/or supplies from the drug cart is maintained in the memory 24 and/or another network-connected terminal such as the pharmacy terminal 42, for example. However, alternate embodiments of the drug cart 56, as shown in FIG. 5, can optionally include a drug cart controller 81 dedicated, or at least specific to that respective cart 56. In other words, when the drug cart controller 81 is connected to the drug cart 56, either via a data cable such as a USB cable or docking station establishing direct communications with circuitry provided to the drug cart 56 or built into the circuitry provided to the drug cart 56 for example, the drug cart controller 81 maintains data concerning the contents and operation of the specific drug cart 56 to which it is provided. The drug cart controller 81 includes much of the same hardware as the computer terminal 10c.

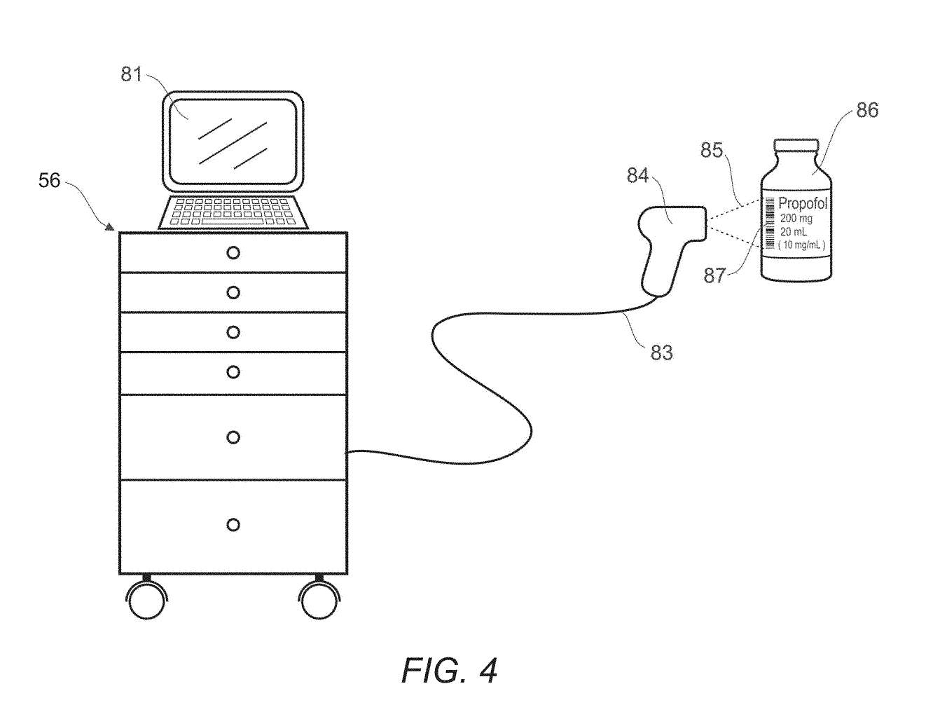

For example, with reference to FIG. 2 for convenience, the drug cart controller 81 can be implemented as a computer including a processing component 22 provided to execute computer-executable instructions stored in a non-transitory, computer-readable memory 24 such as a hard disk drive, read-only memory (“ROM”), random access memory (“RAM”), optical disc, or any other suitable memory device, or any combination thereof, for performing the various functions described herein. The computer-executed instructions, when executed by the computer processor 22, result in the performance of the method of generating a label for a medicinal substance described in detail below. A BIOS 28 is provided to load the operating system and other such administrative instructions 30 stored in the memory 24 and manage hardware interface permissions of the computer terminal 10. The operating system can be configured to only load authorized updates to prevent unauthorized changes to the formulary 36, configuration data 32 and administration instructions 30. Configuration data 32 controls various features of the drug cart 56 (e.g., inventory database, security measures restricting access to drawers and/or specific drugs therein, etc.) that are active and available for use at any given time. A display 14 can also optionally be provided to the drug cart controller 81 to display information to a clinician during use, and a computer-input peripheral such as a non-contact scanner 18 can be provided to interrogate computer-readable codes. Although the internal components of the drug cart controller 81 are described with reference to FIG. 2, using the reference numerals appearing therein, any or all of the specific components can be configured specifically for use in managing the functions of the drug cart 56. For the sake of clarity, components such as the scanner are described and referenced hereinafter using the reference numerals appearing in FIGS. 4-7. Thus, in the description that follows, the scanner 18 is provided to the computer terminal 10c, while the scanner 84 is a hand-held barcode reader or other peripheral scanner provided to the drug cart 56. Embodiments of the drug cart 56 can include a drug cart controller 81 that lacks a hand-held scanner 84 entirely. For such embodiments, the scanner 18 provided to the computer terminal 10c can be utilized to provide such a drug cart 56 with the ability to track drugs dispensed and available inventory based on scans of barcodes 87 during the printing of labels based on the information encoded by those barcodes 87.

With reference to FIG. 4, a drug cart 56 can be configured to include a single hand-held scanner 84 in communication with the drug cart controller 81. Such drug carts 56 may have a single I/O port (e.g., USB port, wireless communication port, etc.) configured to receive a compatible data cable 83 and establish communications between only that single scanner 84 and the drug cart controller 81. The drug cart controller 81 is configured to receive and interpret data indicative of a barcode 87 or other computer-readable code applied to, or otherwise associated with a drug vial 86 removed from the drug cart 56 to identify the drug removed. Handheld scanners 84, however, may be configured with optics and barcode decoding algorithms optimized for reading a barcode on a flat surface, making it difficult to accurately read the barcode 87 applied to a curved surface such as the drug vial 86. Further, the vials 86 can be positioned at various different locations or orientations relative to the read zone 85 of the hand-held scanner 84 each time a barcode 87 is scanned making it more difficult to produce a successful scan. For example, the read zone 85 of the hand-held scanner 84 may encompass a relatively-small area of the vial 86, whereas the scanner 18 provided to the computer terminal 10c includes optics that enlarge the read zone 95 of the scanner 18 to encompass a relatively-large portion of the vial 86. However, the computer terminal 10c can optionally include computer executable instructions or otherwise be configured to correct an optical view of the barcode 87 extending about a curved surface of the vial 86. Additionally, since the scanner 18 of the computer terminal 10c is integrated into the bottom of the display 14 above an opposing portion of the housing 20, the limited space between the scanner 18 and the housing 20 confines the vials 86 to a small region in which the scanner 18 is focused to read barcodes 87. The substantially fixed scanning distance and designated area for resting vial 86 during the scanning process on terminal 10c further enhances the speed and reliability of the scanner to successfully decode barcode 87 because of the predictable location of the barcode 87 relative to the scanner 18. Such design enhancements improve the readability of the barcode 87, thereby making the scanner 18 of the computer terminal 10c more forgiving to the orientation, physical shape and/or position of the vial 86, and more user friendly thereby encouraging compliance by users to scan drug vials. But since the drug cart 56 is typically configured to connect with a single data cable 83, the drug cart 56 may lack the option for connecting a second, different scanner to be used for interrogating the barcode 87 to identify a drug removed from the drug cart 56. And even if a replacement scanner can be installed in place of the hand-held scanner 84, the drug cart controller 81 may not be configured to properly interpret the signals transmitted by such a replacement scanner.

The examples described above describe an inventory monitoring system that relies exclusively on the information from the computer terminal 10. However, some embodiments can utilize information from a plurality of sources to track drug consumption, including excess quantities of drugs that were prepared (e.g., placed in a syringe) but not administered to patients. For example, bolus injections involve the administration of varying quantities of drugs to achieve an effective concentration within the blood. But since the quantity administered to patients may vary, it is not practical to print a label with a predetermined, known quantity of such a drug that is to be administered before a medical procedure begins. The amount of the drug not administered, and remaining in the syringe at the end of the procedure, may be a controlled substance that is to be accounted for. As shown in FIG. 5, the drug cart 56 further includes a shelf 82 on which the computer terminal 10c can be placed. A cable 91 or other suitable (e.g., wireless) communication link can establish communications between the drug cart controller 81 and the computer terminal 10.

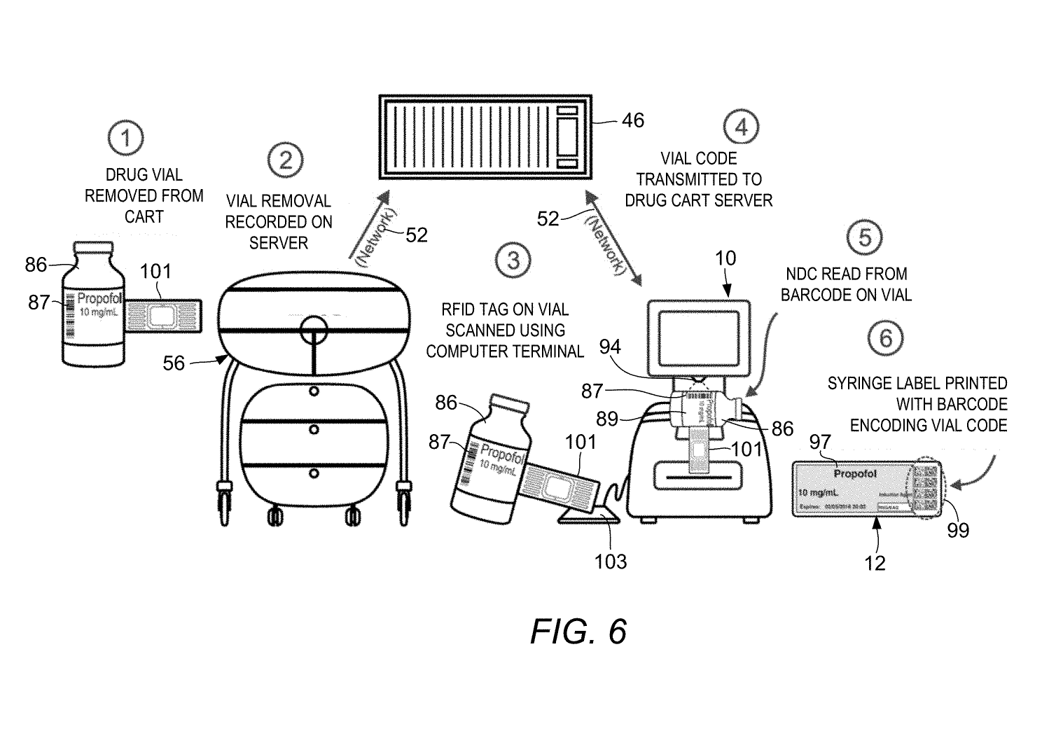

Information obtained via the drug cart 56 can be used by the computer terminal 10 to track a drug's life cycle from the vial 86 to the syringe, and optionally to track waste following administration of the drug. As an example, using the number “135790” as the RFID tag code 102 stored by the RFID tag 101 as shown in FIG. 5, a clinician with the proper credentials can access the drug cart 56 and remove the desired drug vial 86 at step 1 in FIG. 6. The RFID tag reader 104 conducts an occasional, periodical, continuous, or event driven (e.g. closing a drawer 57 on the cart 56), etc. interrogation of the interior of the drawers 57 to determine the vials that remain. Based on the interrogation of the interior of the drawers 57 by the RFID tag reader 104, cart controller 81 can determine that the vial 86 has been removed and transmit the RFID tag code 102 or other data indicative of the vial 86 to the cart server 47 at step 2 in FIG. 6. Since the transmission by the drug cart controller 81 to the server 46 simply indicates the removal of the vial 86 from the drug cart 56, use of the drug remains unknown to the cart server 46 at this point.

As noted above, many workflows require compliance with labeling standards, so the clinician causes the RFID tag 101 to be read by the RFID tag reader 103 as part of the label printing process at step 3. In response to scanning the RFID tag 101 with the RFID tag reader 103 and/or reading the barcode 87 with the barcode reader 94, the RFID tag code 102 can optionally be transmitted by the computer terminal 10 to the cart server 46 via the network 40 to indicate the drug in the removed vial 86 is being used at step 4. The cart server 46 can optionally respond by transmitting information (e.g., drug name, concentration, expiration, etc.) about the drug obtained based on the cart server's formulary to the computer terminal 10 at step 4.

The information obtained by the computer terminal 10 as a result of reading the barcode 87 can be compared to information transmitted by the cart server 46 and received by the computer terminal 10 for validation purposes. Any conflict between the compared information can cause the computer terminal 10 to issue an alert to the clinician, notifying the clinician of this conflict. Alternately, the cart server 46 can compare drug information it has stored that is associated with RFID tag code 102 with drug information received from computer terminal 10 for the same RFID tag code 102 and alert the clinician of conflicts between the compared information.

Continuing with the labeling process, the barcode 87 is read using the barcode reader 94 at step 5. Information obtained as a result of reading the barcode 87 is used by the computer terminal 10 to obtain information from the formulary 36 that is to be included as label content that is to be printed on the label 12 by the printer 26 of the computer terminal 10. For example, information to be included in the label content that is to be printed in human-readable and/or machine-readable form includes at least one of: drug NDC code, drug name, concentration, dose, volume, expiration information indicative of a usable life of the drug in the syringe, designation as a controlled substance, and data indicative of the clinician who prepared the syringe. At least a portion of this information is to be printed in a human-readable format 97 (e.g., using alpha-numeric text) and at least a portion of this information is to be printed in machine-readable format (e.g., a syringe barcode 99) on the label 12. Also encoded by the barcode 99 is the RFID tag code 102 (“135790” according to the present example). Accordingly, subsequently scanning the barcode 12 allows the preparation to be traced back to the specific vial 86 from which the syringe bearing the label 12 was prepared.

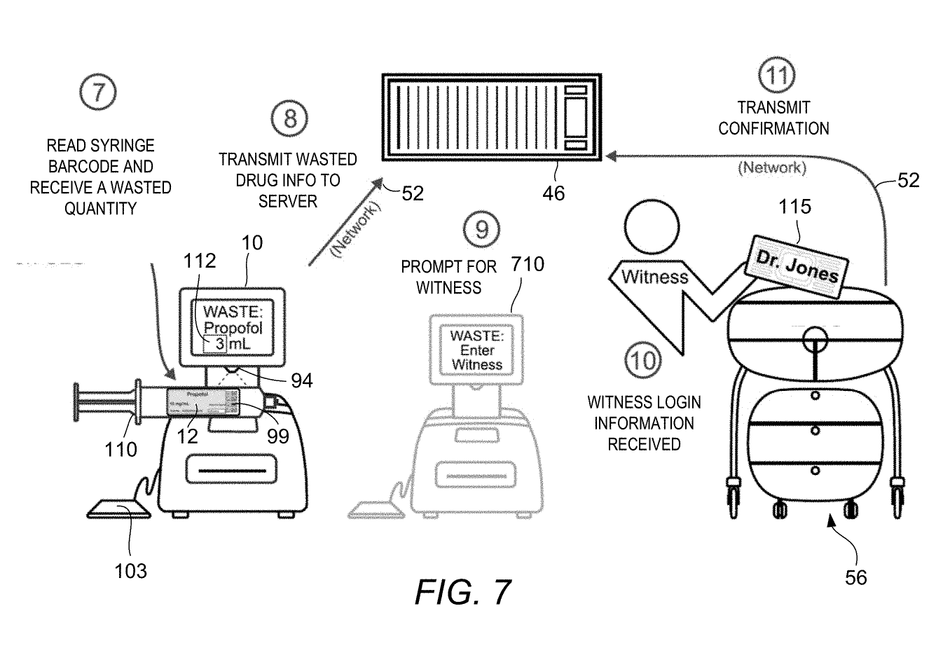

An example method of documenting waste (e.g., an unused quantity of a drug that was prepared for administration) in a syringe 110 can be understood with reference to FIG. 7. At the end of a medical procedure, the syringe barcode 99 on the label 12 provided to the syringe 110 prepared as described above can be read using the barcode reader 94 of the computer terminal 10 to document any “waste” remaining in the syringe 110. In response to reading the syringe barcode 99, the computer terminal 10 extracts the drug information from the barcode 99 and determines whether the drug is a controlled substance, the waste of which must be documented according to a defined protocol. If the drug is determined to be a controlled substance (e.g. formulary has drug identified as a narcotic), the computer terminal 10 will prompt the clinician to enter the amount of the drug being wasted by displaying a waste interface including a fillable quantity field 112 at step 7 in FIG. 7. The computer terminal 10 can optionally be configured to automatically enter “waste” mode in response to scanning a syringe barcode 99 instead of the barcode 87 provided to the vial 86. A clinician who was involved in administering the drug with the syringe 110, or any other clinician, can touch the quantity field 112 to cause a keypad with numerical soft keys suitable for the quantity of the drug being entered to be displayed by the computer terminal 10. The clinician can enter the remaining quantity, which is determined by visually reading the position of the plunger in the syringe 110 relative to gradations marked on the syringe barrel that indicate the amount of fluid in milliliters contained in the syringe. However, this can optionally be an approximate amount or an exact quantity based on a weight of the syringe 110 and a scale operatively connected to the computer terminal 10, for example, and select an “Enter” key, resulting in the entered quantity being transmitted via the network 40 to the cart server 46 at step 8. The transmitted information can include the RFID tag code 102 obtained from the barcode 99 and the quantity of the wasted drug, as specified by the clinician, to the cart server 46. For some embodiments, information about the user/date/time corresponding to the wasting of the drug can optionally also be transmitted.

Some workflows, such as those for narcotics and other controlled substances for example, may require a witness other than the clinician who is wasting the drug to enter confirmation concerning the quantity of the drug being wasted. For such workflows, the cart server 46 can open a record that remains open until pending receipt of the witness confirmation. The computer terminal 10 can be configured to prompt the clinician, at step 9, to have a witness login into the computer terminal 10, drug cart 56, or any other terminal connected to the network 40. Such a witness can optionally be required to login to the drug cart 56 or other network-connected terminal, at step 10 for example, by entering proper login credentials, scanning a RFID tag or other machine readable code provided in the form of a badge 115 issued by the healthcare facility to the witnessing clinician. The drug cart 56 or other network-connected terminal to which the witness logged in can transmit the witness information (e.g., witness name, time of witness confirmation, etc.) to the cart server 46 at step 11 via the network components 52 to complete the waste documentation. Upon receiving valid witness information, the cart server 46 can close the record for this particular syringe 110, and can transmit a signal indicating that the witness procedure has been complete. The computer terminal 10, upon receiving the signal, can clear the prompt on the display requesting witness confirmation.

The above example of documenting waste involves communications with the cart server 46 and optionally a drug cart controller 81. For some embodiments, the computer terminal 10 can extract the drug information from the syringe barcode 99 and, if the drug is a controlled substance (e.g. formulary has drug identified as a narcotic), prompt the user to enter the amount of the drug being wasted in a quantity field 112 similar to that described above. However, the computer terminal 10 will record the amount of the drug being wasted and information about the user/date/time that is wasting the drug. The computer terminal, shown in faded lines at 710 in FIG. 7, will prompt the clinician to have a witness login to the computer terminal 10 to verify the waste amount. Again, the witness will enter authorized user credentials, but using the computer terminal 710 such as by scanning a login badge provided with a barcode compatible with the computer terminal 710 and the barcode reader 94 or an RFID badge compatible with the RFID reader 103. The computer terminal 710 will record the witness information to complete the waste documentation, and will clear the prompt on the display asking for the witness as a result. The computer terminal can optionally transmit this waste transaction to another server connected to the network 40 that will aggregate it with other waste transactions for record keeping purposes.

Accordingly, the computer terminal 10 can optionally record all controlled substances prepared by a given user at preparation time and prompt the user to record the waste from the drugs if any drugs that were not processed by the waste procedure at logout time. The user may be required to process any, and optionally all remaining syringes 110 known to have controlled substances through the waste procedure as a prerequisite to log out from the computer terminal 10.

In another embodiment of the invention, each syringe label printed by computer terminal 10 can have a unique syringe code contained in the machine-readable barcode 99 on the label 12. Such unique syringe code can be generated by the computer terminal 10 or another server on the network 40 and received by computer terminal 10, that is independent of the unique identifying code on RFID tag code 102. The unique syringe code provides traceability of the syringe from drug preparation using computer terminal 10 through administration to the patient and ending with the drug waste process, similar the process previously described for tracking the life cycle of syringes including the RFID tag code 102 in barcode 99 that originated from the RFID tag 101. Although the unique syringe code is independent of the RFID tag code 102, it can optionally be used in conjunction with RFID tag code 102 by configuring computer terminal 10 to include both codes in barcode 99, or configuring terminal 10 to create an association in memory 24 that links of the RFID tag code 102 with the unique syringe code. When such as association exists memory 24, only a single code of the two codes is required in the barcode 99 and computer terminal 10 can look-up the second code when a first code is received by reading barcode 99 using barcode reader 94.

Computer terminal 10 can optionally be configured to allow the unique syringe code to access information stored memory 24 related to that particular syringe that includes for example, the user who prepared the syringe, the date and time it was prepared, the expiration date and time of the drug in the syringe, and information about the drug contained in formulary 36. This information can be provided to other systems or devices on the network 40 in response to properly authorized requests. Alternately, the authorized devices can retrieve the same information from a server on the network 40 that is receiving and aggregating information that includes the unique syringe code from computer terminal 10 devices.

What is claimed is:

1. A method of tracking a drug, the method comprising:
with a code reader operatively connected to a drug labeling device, reading a computer-readable code associated with a drug container used to prepare a delivery container for administering a drug to a patient;
transmitting, over a communication network, a portion of information obtained in response to reading the computer-readable code to a drug server that receives communications from a drug storage device storing an inventory of drugs, the drug container being selectable from the inventory of drugs stored by the drug storage device, wherein the drug server receives drug removal information from the drug storage device during at least one of the communications in response to selection of the drug container from the inventory of drugs;
receiving, over the communication network, at least a portion of the drug removal information transmitted by the drug server, wherein the drug server transmits the portion of the drug removal information as a result of receiving the portion of the information obtained in response to reading the computer-readable code;
obtaining a result of a comparison of information included in: (i) the information obtained in response to reading the computer-readable code, and (ii) the portion of the drug removal information received from the drug server; and
controlling issuance of an alert based on the result of the comparison indicating a conflict exists.
